SEQUENCE LISTING (2 sequences)

SEQ ID NO: 1 - protein, 17 aa, synthetic construct
  CARBOHYD 17: note = C
GPVVPRTQYMSLAFGWX

SEQ ID NO: 2 - protein, 10 aa, synthetic construct
  CARBOHYD 1: note = Ac
  CARBOHYD 10: note = C-NH2
XWKWWPGIFX

(19) United States
(12) Patent Application Publication
(10) Pub. No.: US 2026/0091129 A1
(43) Pub. Date: Apr. 2, 2026

(54) NANOCOMPOSITE FOR TARGETED DEGRADATION OF PATHOGENIC PROTEIN, PREPARATION METHOD THEREFOR, AND USE THEREOF

(71) Applicant: SOUTH CHINA UNIVERSITY OF TECHNOLOGY, Guangzhou (CN)

(72) Inventors: Yunjiao Zhang, Guangzhou (CN); Xiaowan Huang, Guangzhou (CN); Ziyang Cao, Guangzhou (CN); Xianzhu Yang, Guangzhou (CN); Longping Wen, Guangzhou (CN)

(21) Appl. No.: 19/111,784

(22) PCT Filed: Aug. 17, 2023
(86) PCT No.: PCT/CN2023/113588

§ 371 Date: Dec. 12, 2025

(30) Foreign Application Priority Data
Sep. 15, 2022 (CN) 202211119762.4

(51) Int. Cl. A61K 47/69; A61K 9/51; A61K 33/243; A61K 45/06; A61K 47/54; A61K 47/62; A61P 25/28; A61P 35/00; C07K 14/705

(52) U.S. Cl. CPC A61K 47/6937; A61K 9/5123; A61K 9/5153; A61K 9/5192; A61K 33/243; A61K 45/06; A61K 47/543; A61K 47/62; A61P 25/28; A61P 35/00; C07K 14/705

(57) ABSTRACT

Provided are a nanocomposite for targeted degradation of a pathogenic protein, a preparation method therefor, and use thereof, which pertain to the technical field of nanobiological drugs. The nanocomposite for targeted degradation of the pathogenic protein is provided, which comprises a nanocarrier and a protein-targeting binding peptide grafted on the nanocarrier. The nanocarrier is a nanoassembly of maleimide-polyethylene glycol-polylactic acid and cationic lipids; on the other hand, the use of the nanocomposite in the preparation of drugs including an anti-tumor nanodrug and a Huntington's disease inhibiting drug is provided. The nanocomposite can simulate a key receptor protein in a selective autophagy pathway, so that the pathogenic protein to be degraded can be brought into an autophagosome to be degraded by means of an autophagy pathway, thereby effectively solving the problem that PROTACs cannot degrade large-molecular-weight protein aggregates and LYTACs cannot degrade cytoplasmic proteins.

### TECHNICAL FIELD

The present application belongs to a technical field of nanobiological drugs, and in particular relates to a nanocomposite for targeted degradation of a pathogenic protein, a preparation method therefor, and a use thereof.

### BACKGROUND

Abnormal protein homeostasis and accumulation of pathogenic proteins are the causes of various diseases such as tumors and Huntington's disease. Therefore, inducing the degradation of pathogenic proteins and reducing the levels of pathogenic proteins are effective ways for treating related diseases.

Currently, Proteolysis Targeting Chimeras (PROTACs) or Lysosome-Targeting Chimeras (LYTACs) can be used in related technologies to promote the degradation of target proteins through the proteasome pathway or lysosomal pathway.

However, due to the narrow cavity of the proteasome, PROTACs cannot effectively degrade certain large molecular weight proteins or protein aggregates, while LYTACs can only be used to degrade secreted proteins and cell membrane proteins, but cannot degrade cytoplasmic proteins.

### SUMMARY

The embodiments of this application disclose nanocomposite for targeted degradation of a pathogenic protein, a preparation method therefor, and a use thereof, aiming to solve the technical problem that PROTACs cannot effectively degrade large molecular weight pathogenic proteins or protein aggregates, and LYTACs cannot degrade cytoplasmic proteins.

In order to achieve the above purpose, in the first aspect, this application provides a nanocomposite for targeted degradation of a pathogenic protein, the nanocomposite contains a nanocarrier and a pathogenic protein-targeting binding peptide grafted on the nanocarrier. Among them, the nanocarrier is a nano-assembly of maleimide-polyethylene glycol-polylactic acid and cationic lipid.

In the embodiment of the present application, a pathogenic protein-targeting binding peptide is grafted onto a nano-assembly carrier of cationic lipids and maleimide-polyethylene glycol-polylactic acid, so that the nano-assembly carrier and the pathogenic protein-targeting binding peptide cooperate with each other on a basis of respective actions. Specifically, for example, the maleimide-polyethylene glycol-polylactic acid can be combined with the cationic lipids to form a nano-assembly carrier, and the pathogenic protein-targeting binding peptide can be efficiently clicked by “mercapto-maleimide (SH-MAL)” and grafted onto the nano-assembly carrier with a covalent bond to obtain a stable nanocomposite. In particular, based on the fact that cationic lipids can induce significant autophagy effects in cells and promote the formation and accumulation of autophagosomes, we conducted in-depth research on whether the nano-assembly carrier of maleimide-polyethylene glycol-polylactic acid and cationic lipids can increase the number of autophagosomes and be effectively phagocytosed by autophagosomes when taken into cells. The results show that the intervention of maleimide-polyethylene glycol-polylactic acid can further promote the related functions of cationic lipids. Therefore, we grafted the pathogenic protein-targeting binding peptide onto the surface of the nano-assembly carrier to form the nanocomposite, so as to hope to specifically bind to the pathogenic protein and mediate its degradation through the autophagy pathway. The results show that the nanocomposite can effectively bring the pathogenic protein into the autophagosome for efficient degradation, which can effectively solve the problem that PROTACs cannot degrade large molecular weight protein aggregates and LYTACs cannot degrade cytoplasmic proteins. At the same time, further studies have shown that the process of nanocomposite-induced degradation of pathogenic proteins in tumor cells is an autophagy-dependent and ubiquitination-dependent process, and the degradation of pathogenic proteins by nanocomposite can inhibit the gain of function (GOF) of mutant proteins in tumor cells, such as inhibiting the proliferation and migration ability of tumor cells, enhancing tumor cell death, and enhancing the sensitivity of tumor cells to anti-tumor active components. In addition, the nanocomposite not only has good binding with pathogenic proteins, but also can effectively reduce the toxicity to normal cells. It can be used to induce the degradation of different types of pathogenic proteins and has a broad spectrum.

In some embodiments, the preparation method for the nanocarrier in the embodiments of the present application includes:
after dissolving the cationic lipids and maleimide-polyethylene glycol-polylactic acid in an organic solvent, the organic solvent is dripped into the buffer solution and stirred to obtain a nanocarrier solution. Wherein, the nanocarrier solution can be subjected to purification treatment including removing the solvent by rotary evaporation and taking the supernatant after centrifugation. The centrifugation conditions are: centrifugation at a speed of 2000-4000 rpm/min for 3-10 min, specifically centrifugation at a speed of 3000 rpm/min for 5 min.

It should be noted that the organic solvent is preferably tetrahydrofuran, which is beneficial to the dissolution and mixing of maleimide-polyethylene glycol-polylactic acid and cationic lipid; and the buffer is preferably a 0.01M PBS buffer with pH=7.4, so that a normal physiological environment can be simulated to prepare an isotonic nanocarrier solution.

In some embodiments, the cationic lipids include 1,2-dioleoyl-3-trimethylammonium-propane (DOTAP), 2,3-dioleoyloxypropyl-1-trimethylammonium bromide (DOTMA). Among these, 1,2-dioleoyl-3-trimethylammonium-propane is preferred to obtain the best cell autophagy effect.

In some embodiments, the block “polyethylene glycol/polylactic acid” in maleimide-polyethylene glycol-polylactic acid has a molecular weight of 1000-3000. For example, the molecular weight may be 1000, 2000, 3000, etc.

In some embodiments, the mass ratio of cationic lipids to maleimide-polyethylene glycol-polylactic acid is (5-40): 100. Among them, this mass ratio is conducive to improving the level of inducing cell autophagy of the nanocarrier.

In some embodiments, the pathogenic protein is selected from one of mutant p53 protein and mutant huntingtin protein. Among them, the mutant p53 protein contains at least one of the mutation sites of S241F, R175H, R248W, and R280K; the mutant huntingtin protein includes Htt-(Q74).

It should be noted that the source of p53 protein may be cells such as ES-2, MDA-MB-231, MIAPaCa-2 and SK-BR-3; and the source of huntingtin protein may be cells such as PC-12/GFP-Htt(Q74) and Neuro2A/GFP-Htt(Q74).

Notably, when the nanocomposites in the embodiments of the present application are used to degrade mutant p53 protein, they can also effectively inhibit the gain of function (GOF) of the mutant protein in tumor cells, such as inhibiting the proliferation and migration ability of tumor cells, enhancing tumor cell death, and enhancing the sensitivity of tumor cells to active ingredients of drugs.

In some embodiments, the mutant p53 protein-targeting binding peptide has an amino acid sequence of SEQ ID NO. 1; the mutant huntingtin protein-targeting binding peptide has an amino acid sequence of SEQ ID NO. 2. The two targeting binding peptides can efficiently target and bind to the target protein, and the “mercapto-” in the molecular structure can make it efficiently click with maleimide-polyethylene glycol-polylactic acid through “mercapto-maleimide”, so that the targeting binding peptide is efficiently and stably grafted on the nanocarrier.

In the second aspect, the present application provides a preparation method for the nanocomposite, comprising the steps of:
subjecting the pathogenic protein-targeting binding peptide to undergo a click reaction with a solution containing a nanocarrier, and subjecting the reaction product to purification treatment including dialysis, ultrafiltration and concentration, thereby obtaining the nanocomposite, wherein, the dialysis method includes:
after transferring the reaction product to a dialysis bag (MWCO=14000 Da), the reaction product is placed in a PBS solution (pH=7.4, concentration 0.01M) for dialysis.

In some embodiments, the molar ratio of the pathogenic protein-targeting binding peptide to the nanocarrier is (1-3): 1. The molar ratio is conducive to promoting efficient and sufficient loading of the peptide on the surface of the nanocarrier, and improving the capture effect of the target protein.

In the third aspect, the present application provides a use of the nanocomposite in preparation of drugs including anti-tumor nanodrug and Huntington's disease inhibiting drug.

Among them, based on the fact that the nanocomposite has the property of being able to cause pathogenic protein aggregates to be taken into the autophagosome for degradation through the autophagic pathway. Therefore, after the nanocomposite is used to prepare anti-tumor nanodrugs, it can effectively inhibit the proliferation and migration of tumor cells, enhance tumor cell death, and enhance the sensitivity of tumor cells to active ingredients such as cisplatin (CDDP), showing a good synergistic inhibitory effect.

In the fourth aspect, the present application provides an anti-tumor nanodrug, which contains the nanocomposite and an anti-tumor active ingredient(s) loaded on the nanocomposite.

In some embodiments, the anti-tumor active ingredient(s) includes cisplatin, Pt(IV) prodrugs. Among them, the Pt(IV) prodrugs can be prepared according to the literature “Stimuli-responsive clustered nanoparticles for improved tumor penetration and therapeutic efficacy”.

Wherein, loading the anti-tumor active ingredient(s) on the nanocomposite can not only enhance the sensitivity of tumor cells to the anti-tumor nanodrug, but also the anti-tumor nanodrug can show good synergistic anti-tumor effects with patient-derived xenograft (PDX) ovarian cancer.

In the fifth aspect, the present application provides a preparation method for the anti-tumor nanodrug, comprising:
allowing a solution containing maleimide-polyethylene glycol-polylactic acid, cationic lipids and anti-tumor active ingredient(s) to self-assemble and dialyze in a buffer solution to obtain a solution containing drug-loaded nanoparticles;
allowing the solution containing drug-loaded nanoparticles to undergo a click reaction with a pathogenic protein-targeting binding peptide, and dialyzing, ultrafiltering and concentrating the reaction product in sequence to obtain an anti-tumor nano drug.

Among them, the dialysis in first step can effectively remove unreacted anti-tumor active ingredients; and the dialysis in second step can effectively remove unreacted peptides.

Compared with the prior art, the advantages or beneficial effects of the embodiments of the present application at least include:

According to the nanocomposite in the embodiment of the present application, by grafting the pathogenic protein-targeting binding peptide onto the nanoassembly carrier co-assembled by cationic lipids and maleimide-polyethylene glycol-polylactic acid, on the one hand, the nanocomposite can simulate the key receptor protein in the selective autophagy pathway, so that the pathogenic protein is brought into the autophagosome and degraded through the autophagy pathway, which can effectively solve the problem that PROTACs cannot degrade large molecular weight protein aggregates and LYTACs cannot degrade cytoplasmic proteins. On the other hand, the nanocomposite can form good binding with a variety of pathogenic proteins and effectively reduce toxicity to normal cells, and it can be used to induce degradation of different types of pathogenic proteins, and has a broad spectrum.

### BRIEF DESCRIPTION OF DRAWINGS

FIG. 1 is a structural schematic view of NRs;

FIG. 2 is a fluorescence image of the autophagy effect of DOTAP-induced MDA-MB-231-EGFP-LC3 cells;

FIG. 3 is a western blot image of the degradation of mutant p53 protein by nanocomposite containing neutral lipid, anionic lipid and cationic lipid;

FIG. 4 is a particle size distribution graph and Zeta potential graph of mNRs, dNRs and NRs;

FIG. 5 is a particle size distribution graph of mNRs, dNRs and NRs in a PBS solution containing 10% FBS;

FIG. 6 is a transmission electron microscopy image of mNRs, dNRs and NRs;

FIG. 7 is a circular dichroism spectrum of mutant p53 protein after treatment with PBS, MBP, dNRs and NRs;

FIG. 8 is an SPR spectrum of mutant p53 protein after different treatments;

FIG. 9 is a STORM electron microscope image of NRs;

FIG. 10 is a western blot image of NRs containing different concentrations of DOTAP degrading mutant p53 protein in ES-2 cells;

FIG. 11 shows a cell viability of NRs containing different concentrations of DOTAP in HEK 293T cells;

FIG. 12 is an immunofluorescence image of MBP, mNRs, dNRs and NRs degrading mutant p53 protein in ES-2 cells;

FIG. 13 is a graph of mRNA changes of p53 in ES-2 cells after NRs treatment;

FIG. 14 is a western blot image of NRs degrading mutant p53 protein in ES-2, MDA-MB-231, MIAPaCa-2 and SK-BR-3 cells;

FIG. 15 is a western blot image for detecting the specific degradation pathway of NRs in ES-2 cells to degrade mutant p53 protein;

FIG. 16 is a western blot image for detecting the ubiquitination dependence of NRs in the process of degrading mutant p53 protein in ES-2 cells;

FIG. 17 is a biological electron microscopy image showing that NRs induces autophagic effect in ES-2 cells;

FIG. 18 is a fluorescence image of complete autophagy induced by NRs in ES-2 cells;

FIG. 19 is a western blot image of the degradation of mutant p53 protein induced by NRs in ES-2 cells requiring the participation of ubiquitinase;

FIG. 20 is a western blot image of increased ubiquitination levels induced by NRs in ES-2 cells;

FIG. 21 shows the cell viability of HUVEC, H1299, HEK 293T, A549, HCT 116, MIAPaCa-2, SK-BR-3, MDA-MB-231 and ES-2 after NRs treatment;

FIG. 22 is a graph showing the sphering experiments of MBP, mNRs, dNRs and NRs in ES-2 cells;

FIG. 23 is a graph showing the Transwell results of MBP, mNRs, dNRs and NRs in ES-2 cells;

FIG. 24 is a Hoechst/PI double staining image of MBP, mNRs, dNRs and NRs in ES-2 cells;

FIG. 25 is a graph of the biodistribution of DiD-NRs in ES-2 tumor-bearing mice;

FIG. 26 is a graph of the in vivo treatment experiment of nanoparticles in ES-2 tumor-bearing mice;

FIG. 27 is a western blot image of p53 in tumors of each group of mice in ES-2 tumor-bearing mice;

FIG. 28 is an immunofluorescence graph of p53 and LC3 in tumor sections of each group of mice in ES-2 tumor-bearing mice;

FIG. 29 is a graph of the in vivo treatment experiment of nanoparticles in PDX model mice;

FIG. 30 is a western blot image of p53 in tumors of each group of mice in PDX model mice;

FIG. 31 is an immunohistochemistry and TUNEL staining graph of p53 in tumor sections of each group of mice in PDX model mice;

FIG. 32 is a western blot image showing that NRs-Htt induces cell autophagy effect and degrades GFP-Htt (Q74) protein in PC-12/GFP-Htt (Q74) and Neuro 2A/GFP-Htt (Q74) cells;

FIG. 33 is an immunofluorescence image showing that NRs-Htt degrades GFP-Htt (Q74) protein in PC-12/GFP-Htt (Q74) and Neuro 2A/GFP-Htt (Q74) cells.

### DETAILED DESCRIPTION

The following will be combined with the drawings in the embodiments of the present application to clearly and completely describe the technical solutions in the embodiments of the present application. Obviously, the described embodiments are part of the embodiments of the present application, not all of the embodiments. Based on the embodiments in the present application, all other embodiments obtained by ordinary technicians in this field without creative work are within the scope of protection of this application.

### Example 1

This example provides a preparation method for nanocomposite NRs for targeting mutant proteins, which specifically includes:

S101—Preparation of nanocarrier dNRs:

10 mg of Mal-PEG2000-PLA2000 and 2.0 mg of cationic lipid DOTAP were dissolved in 1.2 mL of tetrahydrofuran and transferred to a 5 mL EP tube, vortexed and mixed evenly to obtain a mixed solution;

Under stirring, the mixed solution was dripped dropwise into 10 ml of PBS (pH=7.4, 0.01 M, the same below), and continued to stir at room temperature for 2 hours to obtain a nanoparticle solution;

The tetrahydrofuran in the nanoparticle solution was removed by a rotary evaporator, and after centrifugation at a speed of 3000 rpm/min for 5 min, the supernatant was taken to obtain a nanocarrier dNRs solution (NPs@DOTAP);

### S102—Preparation of Nanocomposite NRs:

5.0 mg of the MBP peptide shown in SEQ ID NO.1 was added to the dNRs solution, and the reaction was stirred at room temperature for 8 hours to obtain the impurity product NRs (MBP-NPS@DOTAP);

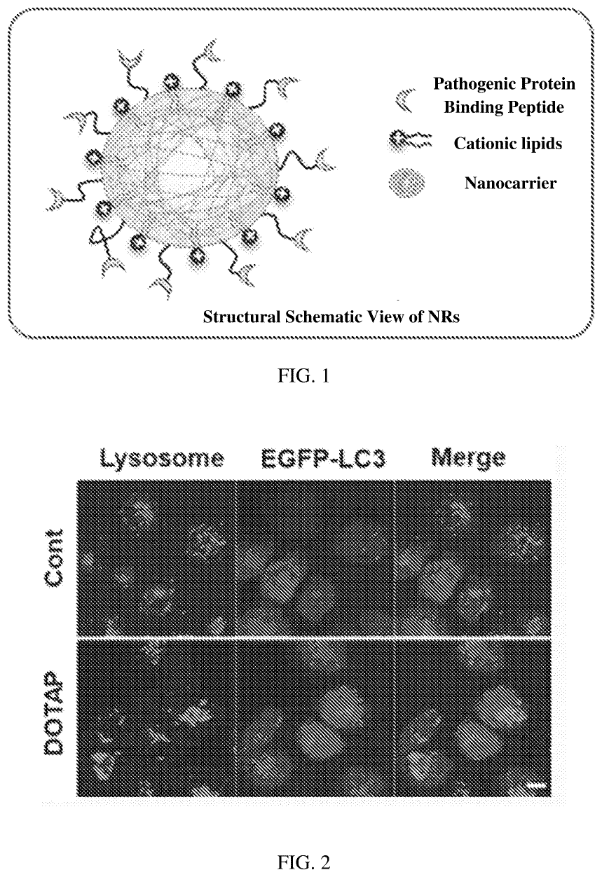

The impurity product NRs was transferred to a dialysis bag (MWCO=14000 Da), and dialyzed in 2 L of PBS solution overnight, and then the NRs solution was ultrafiltered and concentrated by AmiconYM-30 centrifugal filter device (MWCO=5000 Da) to obtain the purified nanocomposite NRs (MBP-NPS@DOTAP) shown in FIG. 1. FIG. 1 is a structural schematic view of NRs.

It can be seen from FIG. 1 that the nanocomposite is composed of self-assembled carrier particles of Mal-PEG2000-PLA2000 and cationic lipids and pathogenic protein binding peptides modified on the surface of the self-assembled carrier particles.

In this example, DOTAP was replaced by neutral lipids (GTP, PL), anionic lipids (DPPG, DOPG) and cationic lipids (DOTMA) respectively to prepare the nanocomposites NPs@GTP, NPs@PL, NPs@DPPG, NPs@DOPG and NPs@DOTMA.

In this example, the amount of DOTAP added was adjusted, and the nanocomposites mNRs (NRs@0% DOTAP), NRs@5% DOTAP, NRs@10% DOTAP, NRs@20% D-OTAP and NRs@40% DOTAP were prepared referring to the method of Example 1.

In addition, in this example 1, 5.0 mg of the HBP peptide shown in SEQ ID NO.2 was added to the dNRs solution to prepare the nanocomposite NRs-Htt (HBP-NPs@DOTAP).

### Example 2

This example provides a preparation method for anti-tumor nanodrug NRs/Pt(IV), which specifically includes:

S201: 10 mg of Mal-PEG2000-PLA2000 and 2.0 mg of cationic lipid DOTAP were dissolved in 1.2 mL of tetrahydrofuran, and 1 mg of Pt(IV) prodrug (DMSO, 10 mg/mL) was added, and then transferred to a 5 mL EP tube, and then vortexed and mixed evenly to obtain a drug-loaded mixed solution;

S202: Under stirring, the drug-loaded mixed solution was dripped dropwise into 10 mL of PBS, and stirring was continued at room temperature for 2 hours to obtain a drug-loaded nanoparticle solution;

S203: The tetrahydrofuran in the drug-loaded particle solution was removed by a rotary evaporator and transferred to a dialysis bag (MWCO=14000 Da) for dialysis for 8 hours to obtain nanoparticles dNRs/Pt(IV);

S204: The MBP peptide (5.0 mg, 0.0025 mM) was added to the dNRs/Pt(IV) solution and stirred at room temperature for 8 hours to obtain impure NRs/Pt(IV);

S205: After transferring NRs/Pt(IV) to a dialysis bag (MWCO=14000 Da), it was dialyzed in 2.0 L of PBS solution overnight, and the drug-loaded nanoparticles were ultrafiltered and concentrated by an AmiconYM-30 centrifugal filter device to obtain purified NRs/Pt(IV).

Various nanocomposites were tested. The chemical reagents and biological raw materials used in the test were commercially available, and the reagent concentrations and overnight cultures were in accordance with conventional requirements, such as overnight culture (37° C., 5% CO₂).

1. Test of the ability of cationic lipids to induce cell autophagy

After MDA-MB-231 cells were transfected with EGFP-LC3 plasmid to obtain MDA-MB-231-EGFP-LC3 cells, they were seeded in a 24-well cell culture plate with a circular slide (density of about 3×10⁴/well), cultured overnight, and PBS and DOTAP (2 μg/mL) were added respectively. After 6 hours of culture, fluorescence detection was performed, and the results are shown in FIG. 2. FIG. 2 is a fluorescence image of the autophagy effect of DOTAP-induced MDA-MB-231-EGFP-LC3 cells.

It can be seen from FIG. 2 that after DOTAP treatment, the LC3 green fluorescent protein in the cells showed obvious dot-like aggregation, which proved that DOTAP can induce significant autophagy effects in cells.

2. Comparison of the ability of nanocomposites containing neutral lipids, anionic lipids and cationic lipids to induce cell autophagy respectively

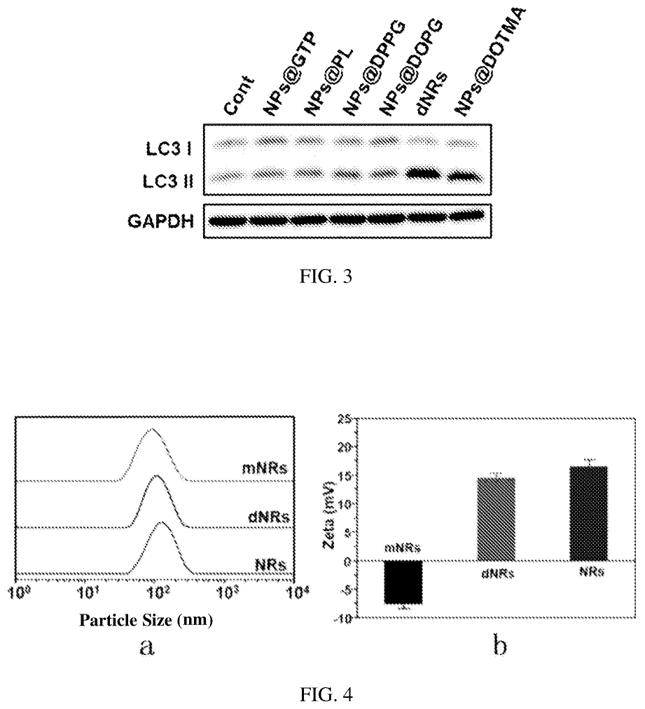

MDA-MB-231-EGFP-LC3 cells were seeded in a 24-well cell culture plate (density of about 3×10⁴/well), cultured overnight, and NPs@GTP, NPs@PL, NPs@DPPG, NPs@DOPG, NPS@DOTMA and NPs@DOTAP (dNRs) were added respectively. After 6 hours of culture, the LC3 protein level in the cells was detected by western blot. The results are shown in FIG. 3. FIG. 3 is a western blot image of the degradation of mutant p53 protein by nanocomposite containing neutral lipids, anionic lipids and cationic lipids.

It can be seen from FIG. 3 that there was no further accumulation of LC3-II protein in cells treated with nanocomposites containing neutral lipids (GTP, PL) and anionic lipids (DPPG, DOPG), while the LC3-II protein in cells treated with nanocomposites containing cationic lipids (DOTMA, DOTAP) increased significantly, and the increase of LC3-II protein in cells treated with nanocomposites containing DOTAP was the most obvious. It was proved that nanocomposites containing cationic lipids can induce significant autophagy effects in cells, and the DOTAP-induced autophagy effects are the best.

3. Determination of particle size and surface potential of mNRs, dNRs and NRs

After mNRs, dNRs and NRs were dispersed in ultrapure water at a concentration of 1.0 mg/mL, the particle size and surface potential were measured with a nanoparticle size analyzer, and the results are shown in FIG. 4. FIG. 4a is the particle size distribution graph of mNRs, dNRs and NRs; FIG. 4b is the Zeta potential graph of mNRs, dNRs and NRs.

It can be seen from FIG. 4a that the particle sizes of dNRs, mNRs and NRs were all around 100 nm; it can be seen from FIG. 4b that mNRs are electronegative, and the potential of dNRs containing DOTAP was strongly positive, and its potential was about +15 mV. This is because the component contains the positively charged cationic lipid DOTAP. In addition, NRs also showed strong surface electropositivity.

4. Stability characterization of mNRs, dNRs and NRs

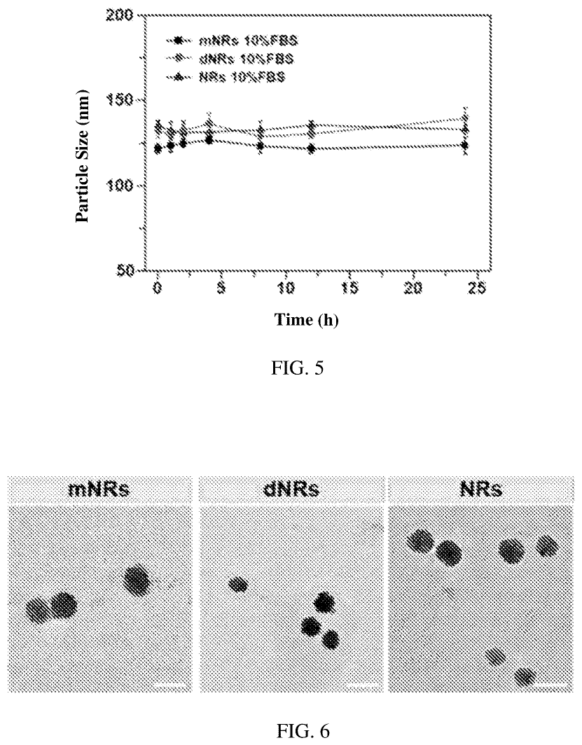

mNRs, dNRs and NRs were dispersed in PBS solution containing 10% fetal bovine serum (FBS) at a concentration of 1.0 mg/mL, and were incubated at 37° C. for 0, 1, 2, 4, 8, and 12 and 24 h, respectively. The particle size was measured with a nanoparticle size analyzer, and the results are shown in FIG. 5. FIG. 5 is the particle size distribution graph of mNRs, dNRs and NRs in PBS solution containing 10% FBS.

It can be seen from FIG. 5 that the particle size of mNRs, dNRs and NRs showed no significant change within 24 hours, indicating that mNRs, dNRs and NRs have good particle stability in PBS solution containing 10% FBS.

5. Morphological characterization of mNRs, dNRs and NRs

10 μL of mNRs, dNRs or 1.0 mg/mL NRs solution were dropped in a 400-mesh copper mesh and placed in a clean and dry place to dry overnight, respectively. The morphology was then observed using a transmission electron microscope. The results are shown in FIG. 6. FIG. 6 is the transmission electron microscope image of mNRs, dNRs and NRs.

It can be seen from FIG. 6 that mNRs, dNRs and NRs all showed complete spherical morphology under the electron microscope, and their sizes were all around 100 nm.

6. Detection of the binding of NRs and mutant proteins by circular dichroism spectroscopy

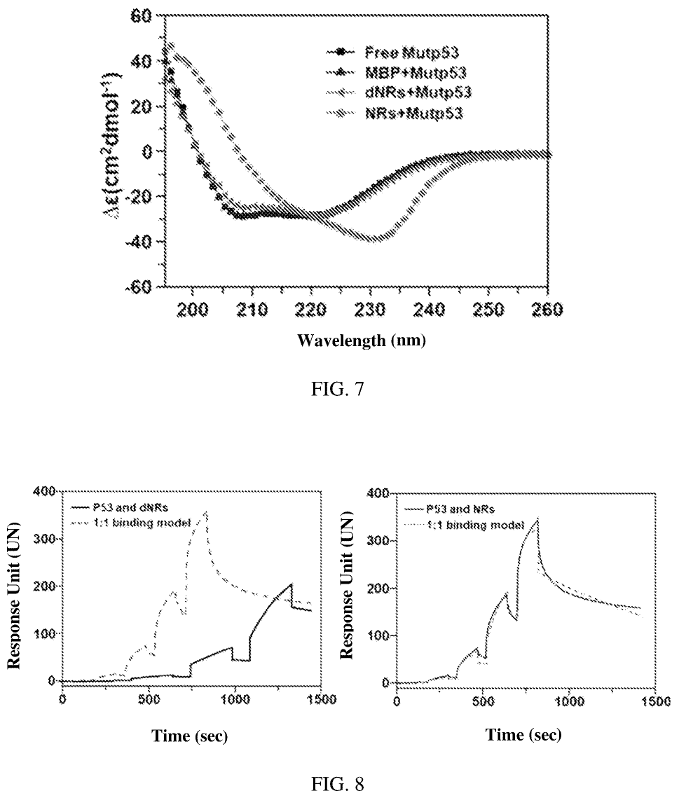

1.25 mM mutant p53S241F protein (Mutp53) solution was taken, and an equal volume of PBS and MBP peptide (1.25 mM), dNRs (5 mg/mL) or NRs (5 mg/mL) were added and mixed, and then stirred for 2 hours at room temperature, and then each group of the mixed solutions was transferred to quartz cuvettes, and the circular dichroism spectrum of the mutant p53 protein was detected by a Chirascan circular dichroism spectrometer (spectral conditions: temperature was 65° C.; the spectrum detection range was 195-260 nm), and the results are shown in FIG. 7. FIG. 7 is the circular dichroism spectrum of the mutant p53 protein after treatment with PBS, MBP, dNRs and NRs.

It can be seen from FIG. 7 that in the group with Mutp53 alone and the experimental group incubated with dNRs and Mutp53, the spectrum of the mutant p53 protein showed two negative peaks at 208 nm and 222 nm, which correspond to the secondary structure of the mutant p53 protein; while the spectrum of the mutant p53 after incubation with NRs showed a minimum peak at 231 nm, indicating that the secondary structure of the mutant p53 protein had undergone a significant transformation. It was proved that NRs can bind to the mutant p53 protein, thereby causing changes in its secondary structure.

7. Detection of the binding of NRs and mutant proteins by surface plasmon resonance (SPR) method

A GE BIAcore 8K instrument was operated at a constant temperature of 25° C. Each CM5 sensor chip consisted of 8 identical experimental channels, each of which was divided into two flow cells (flow-cell 1 and fow-cell 2). Flow-cell 1 (Fc1) was always kept blank as a reference, while fow-cell 2 (Fc2) was used to study the interaction between NRs and mutant p53 proteins.

Firstly, the system was balanced with PBS-T buffer (20 mM Na-phosphate, 150 mM NaCl, 0.05% Tween 20, pH=7.4); secondly, the experimental channel was rinsed with a mixed solution of EDC (0.2 M) and NHS (0.05 M) for 6 min to activate the carboxyl groups on the sensor; thirdly, NRs (10 mM acetic acid buffer, pH=4.0) were injected into the Fc2 cell for 7 min to immobilize NRs on the sensor chip through reaction between the amino group of the peptide pairs on the NRs surface and carboxyl group; finally, 1 M ethanolamine-HCl solution was injected into the Fc1 and Fc2 cells respectively to block the remaining carboxyl active esters.

To observe the interaction between the mutant p53 protein (p53S241F) and NRs, 5.21 UM mutant p53 protein solution was injected onto the NRs-modified sensor surface. The injection time was 3 min, the flow rate was 30 μL/min, and then there was a 5 min dissociation step. In order to calculate the binding constant (Kd value), under the same conditions, the mutant p53 proteins with concentrations of 2500 nM, 500 nM, 100 nM, 20 nM and 4 nM were injected into the channel, and the binding signal was measured in each cycle. Finally, the binding kinetic constant between NRs and mutant p53 protein was calculated using the BIAcore 8K Evaluation software with a 1:1 binding model. The results are shown in FIG. 8. FIG. 8 is the SPR spectrum of mutant p53 protein after different treatments.

It can be seen from FIG. 8 that the binding isotherm between mutant p53S241F protein and NRs conformed to the standard 1:1 binding model, and NRs can effectively bind to mutant p53 at various protein concentrations. Further, the Kd value of the interaction between mutant p53 protein and NRs was determined to be 1.40×10⁻⁹ by single-cycle kinetics. In contrast, the binding efficiency between dNRs without MBP peptide modification and mutant p53 was much lower than that between NRs and mutant p53, indicating that NRs can effectively bind to mutant p53 through specific high-affinity interactions.

8. Detection of the binding of NRs nanoparticles and mutant proteins by stochastic optical reconstruction microscopy (STORM)

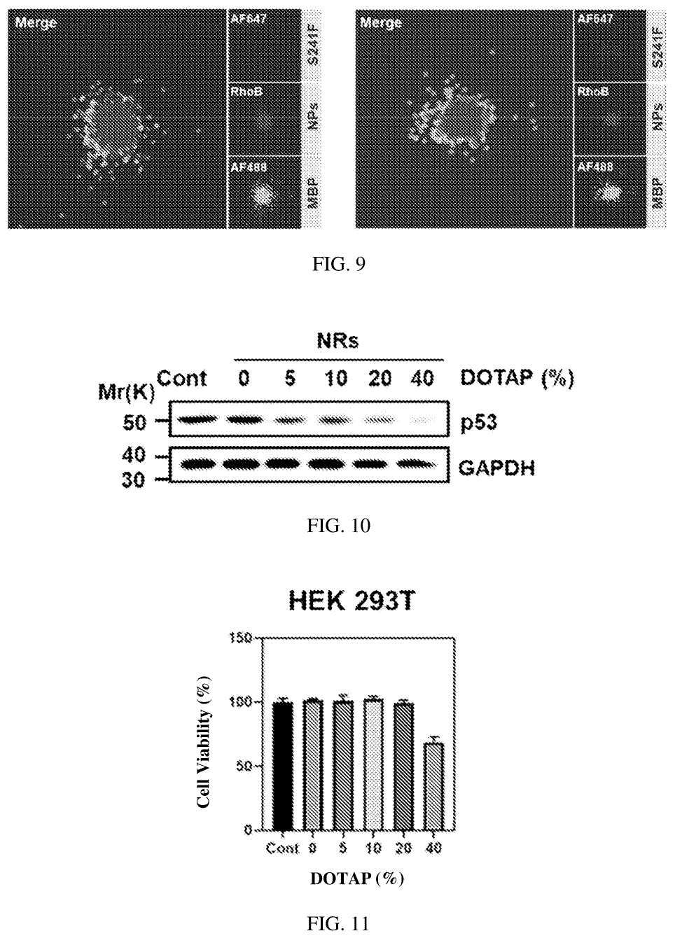

In order to visually observe the modification of MBP peptide on the surface of NRs and the binding of mutant p53 protein (p53S241F) to NRs, referring to the preparation process of Example 1, Rhodamine B (RhoB)-labeled PEG-b-PLA (RhoB-PEG-b-PLA) was added during the preparation of NRs to obtain RhoB (red fluorescent)-labeled nanoparticles; Aleax Fluor™ 488-NHS-labeled MBP was added via click reaction to obtain the dual fluorescent NRs of MBP whose the core was labeled with RhoB and the surface was labeled with Aleax Fluor™ 488; Aleax Fluor™ 647-NHS-labeled mutant p53 protein was added to the dual fluorescent NRs, stirred and mixed at room temperature for 4 hours, and then transferred to a dialysis bag (MWCO=14000 Da) for dialysis to remove unreacted MBP peptide; the mixed solution of fluorescently labeled NRs and mutant p53 protein was added to a glass bottom culture dish, allowed to settle for 10 min, and fluorescence images of 488 nm, 530 nm and 647 nm channels were collected using a stochastic optical reconstruction microscopy, and the results are shown in FIG. 9. FIG. 9 is a STR electron microscope image of NRs.

It can be seen from FIG. 9 that obvious green fluorescence was observed around the RhoB (red fluorescent)-labeled NPs, indicating that the MBP peptide was successfully modified on the surface of NRs. After the Alexa Fluor™ 647-labeled mutant p53 protein was mixed with the NRs solution, the blue fluorescence of the mutant p53 protein and the green fluorescence of the MBP peptide had a more obvious co-localization phenomenon and were dispersed around the NRs, indicating that NRs can effectively bind to mutant p53 protein through the high-affinity interaction between MBP peptide and mutant p53 protein.

9. Degradation experiment of mutant p53 protein induced by NRs

### 9.1 Evaluation of the Ability of NRs Containing Different Concentrations of DOTAP to Clear Mutant p53 Protein

ES-2 cells were seeded in a 24-well cell culture plate (density approximately 5×10⁴/well) and cultured overnight. PBS and NRs containing different concentrations of DOTAP (0%, 5%, 10%, 20% and 40%) were added respectively. After culturing for 12 hours, western blot detection was performed. The results are shown in FIG. 10. FIG. 10 is the western blot image of NRs containing different concentrations of DOTAP degrading mutant p53 protein in ES-2 cells.

It can be seen from FIG. 10 that as the concentration of DOTAP increased, the effect of NRs in clearing mutant p53 protein in ES-2 cells was gradually enhanced.

9.2 Evaluation of the toxicity of NRs containing different concentrations of DOTAP to normal cells

HEK 293T cells were seeded in a 96-well cell culture plate (density was approximately 1×10⁴/well) and cultured overnight. PBS and NRs containing different concentrations of DOTAP (0%, 5%, 10%, 20% and 40%) were added. After culturing for 24 hours, MTT detection was performed. The results are shown in FIG. 11. FIG. 11 shows the cell viability of NRs containing different concentrations of DOTAP in HEK 293T cells.

It can be seen from FIG. 11 that compared with other groups, the cell survival rate of NRs containing 40% DOTAP in HEK 293T cells was significantly reduced, indicating that NRs containing 40% DOTAP are more toxic to normal cells. So we all chose NRs containing 20% DOTAP, which are less toxic and better at clearing mutant p53 protein, for the subsequent experiments.

9.3 Evaluation of the ability of free MBP peptide, mNRs, dNRs and NRs to clear mutant p53 protein

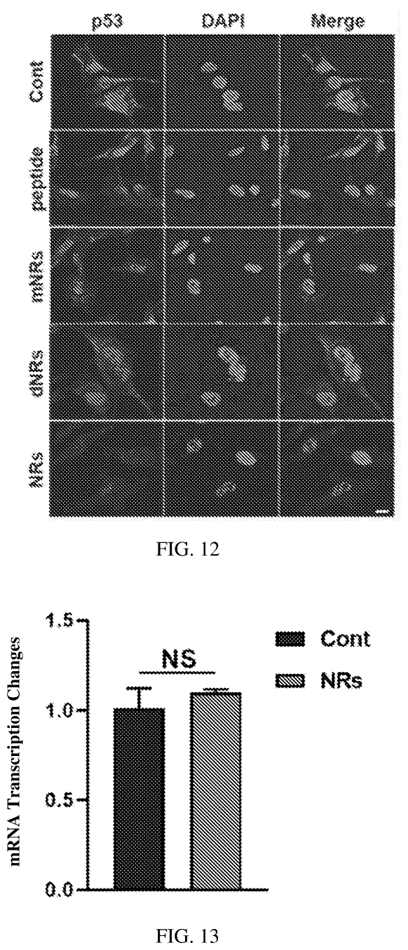

ES-2 cells were seeded in a 24-well cell culture plate (density approximately 3×10⁴/well) with circular slides placed in advance, and cultured overnight. PBS, free MBP peptide (250 g/mL), and mNRs, dNRs, and NRs all at a concentration of 500 μg/mL were added respectively. After 6 hours of culture, immunofluorescence detection was performed. The results are shown in FIG. 12. FIG. 12 is the immunofluorescence image of MBP, mNRs, dNRs and NRs degrading mutant p53 protein in ES-2 cells.

It can be seen from FIG. 12 that compared with other groups, the red fluorescence of labeled mutant p53 protein in the NRs-treated group was significantly reduced. It was proved that NRs can effectively reduce the mutant p53 protein level in ES-2 cells.

9.4 Testing whether NRs affect the mRNA levels of mutant p53

ES-2 cells were seeded in a 6-well cell culture plate (density approximately 3×10⁵/well) and cultured overnight. PBS and NRs (500 μg/mL) were added respectively. After 12 hours of culture, RNA was extracted using TRIzol RNA extraction reagent (Invitrogen), and the mRNA level of p53 was detected using RT-PCR. The results are shown in FIG. 13. FIG. 13 is a graph showing changes in mRNA of p53 in ES-2 cells after NRs treatment.

It can be seen from FIG. 13 that no significant difference in mutant p53 mRNA levels was observed between the NRs-treated group and the PBS control group, confirming that NRs clear the mutant p53 protein in ES-2 cells through the degradation pathway.

9.5 Testing the broad spectrum of NRs for degradation of different types of mutant p53

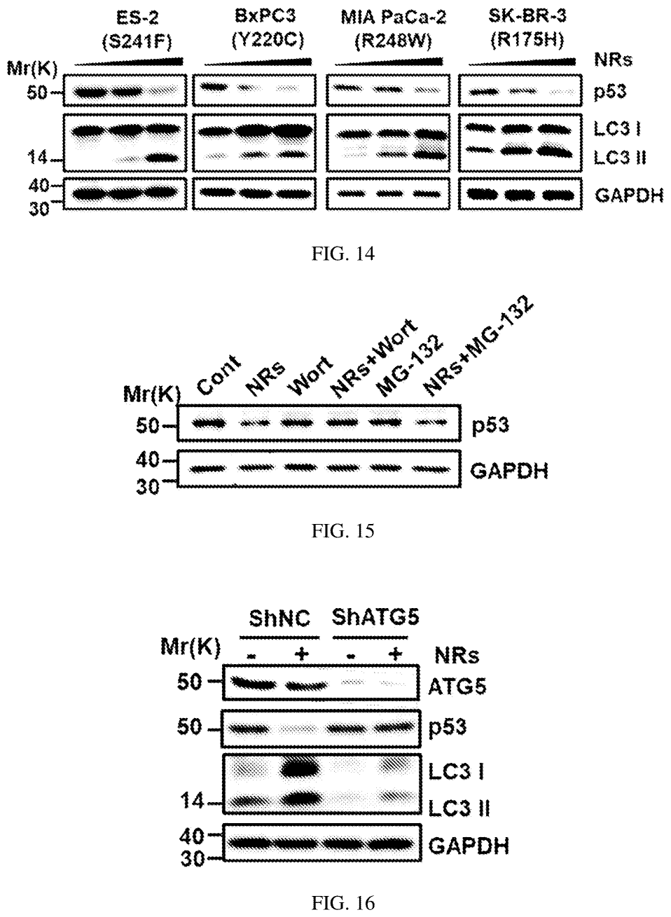

ES-2, MDA-MB-231, MIAPaCa-2 and SK-BR-3 cells were seeded in a 24-well cell culture plate (density approximately 5×10⁴/well) and cultured overnight. PBS and 250 μg/mL and 500 μg/mL NRs were added respectively. After 12 hours of culture, the level of mutant p53 protein was detected by western blot. The results are shown in FIG. 14. FIG. 14 is the western blot image of NRs degrading mutant p53 protein in ES-2, MDA-MB-231, MIAPaCa-2 and SK-BR-3 cells.

It can be seen from FIG. 14 that as the concentration of NRs increases, the mutant p53 proteins in the four tumor cells containing different p53 mutation sites gradually decrease. It was proved that NRs can degrade many different types of mutant p53 and has a certain broad spectrum.

10. NRs induce degradation of mutant p53 protein in an autophagy-dependent and ubiquitination-dependent manner
10.1 Testing the Pathway by which NRs Degrade Mutant p53 Protein in Cells

ES-2 cells were seeded in a 24-well cell culture plate (density approximately 5×10⁴/well) and cultured overnight. PBS, 500 μg/mL NRs, autophagy inhibitor Wortmannin (abbreviated as Wort, 1 μM), 500 μg/mL NRs+Wort, proteasome inhibitor MG-132 (10 μM), and 500 μg/mL NRs+MG-132 (10 μM) were added respectively. After 12 hours of culture, western blot detection was performed, and the results are shown in FIG. 15. FIG. 15 is a western blot image for detecting the specific degradation pathway of NRs in ES-2 cells to degrade mutant p53 protein.

It can be seen from FIG. 15 that the autophagy inhibitor Wortmannin can effectively inhibit the degradation of mutant p53 protein triggered by NRs, but the proteasome inhibitor MG-132 cannot. This indicates that the degradation of mutant p53 protein triggered by NRs is mediated through the autophagy pathway.

10.2 Testing the autophagy dependency of NRs degrading mutant p53

HEK293T cells were seeded in a 6-well cell culture plate (density of about 3×10⁵/well) and cultured overnight. The control shRNA (shNC) and shATG5 for knocking out ATG5 and the lentiviral vector system were co-transfected into HEK 293T cells by using lipofectamine 3000. After 48 hours of transfection, the viral supernatant was collected to infect ES-2 cells. 24 hours later, the virus-infected ES-2 cells were seeded in a 24-well plate, and treated with PBS or 500 μg/mL NRs for 12 hours, respectively. Western blot detection was performed, and the results are shown in FIG. 16. FIG. 16 is a western blot image for detecting the autophagy dependency of NRs in the process of degrading mutant p53 protein.

It can be seen from FIG. 16 that compared with the control shNC group, the expression level of ATG5 protein in ES-2 cells was significantly reduced after lentivirus infection, and after knocking out the autophagy-related protein ATG5, the degradation of mutant p53 protein triggered by NRs was significantly inhibited. It was proved that the degradation of mutant p53 protein by NRs is autophagy-dependent.

10.3 Testing the ability of NRs to induce cell autophagy

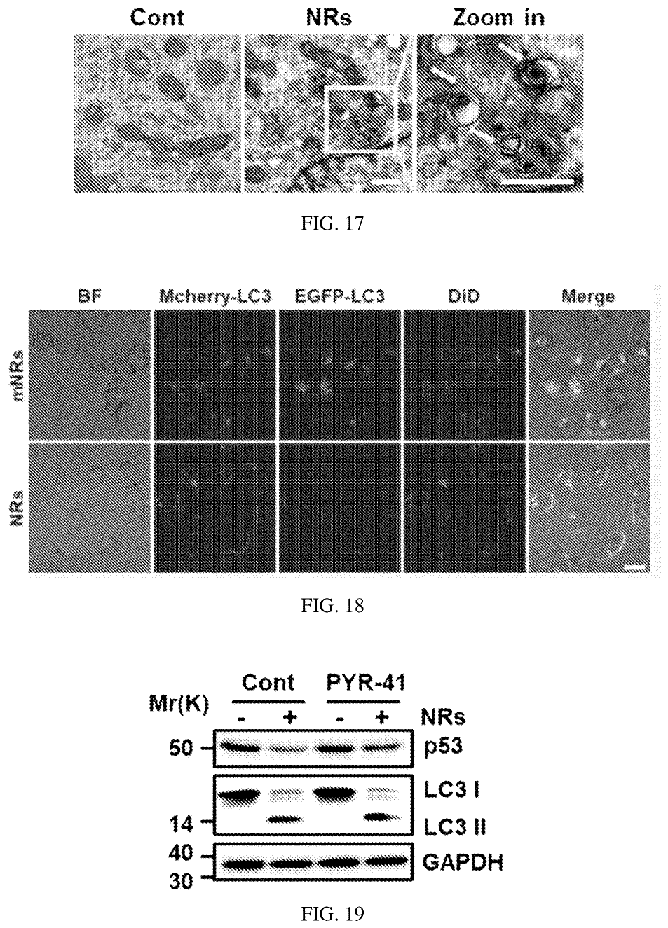

ES-2 cells were seeded in a 10 mm cell culture dish (density approximately 5×10⁶/dish) and cultured overnight. PBS and NRs with a concentration of 500 μg/mL were added, respectively. After 12 hours of culture, the cell pellets were collected and fixed overnight at 4° C. by using electron microscopy fixative. The cells were then embedded and cut into ultrathin sections, stained with uranyl acetate and lead citrate, and bioelectron microscopy was used to observe the autophagosomes in the cytoplasm. The results are shown in FIG. 17. FIG. 17 is a bioelectron microscope image of NRs triggering autophagy effect in ES-2 cells.

It can be seen from FIG. 17 that the number of autophagosomes increased significantly in ES-2 cells treated with NRs. It was proved that NRs can effectively induce autophagy effect in cells.

10.4 Testing the integrity of cell autophagy induced by NRs

MDA-MB-231 cells were transfected with EGFP-mCherry-LC3 plasmid to obtain MDA-MB-231-EGFP-mCherry-LC3 cells, which were seeded in a 24-well cell culture plate (density is about 3×10⁴/well) with a circular slide placed in advance, and cultured overnight. DiD-mNRs and DID-NRs at a concentration of 500 μg/mL were added (wherein, referring to the method of mNRs and NRs in Example 1, DiD fluorescent dye was added during preparation to obtain DiD-labeled DiD-mNRs and DiD-NRs), respectively. After culturing for 12 hours, fluorescence detection was performed, and the results are shown in FIG. 18. FIG. 18 is a fluorescence image of complete autophagy induced by NRs in ES-2 cells.

It can be seen from FIG. 18 that in the cells treated with mNRs, mCherry (red fluorescence) and EGFP (green fluorescence) expressing the LC3 protein showed obvious co-localization; whereas in the cells treated with NRs, EGFP fluorescence was easily quenched under the acidic conditions of autophagic lysosomes, so the green fluorescence was significantly reduced, while the red fluorescence was relatively stable, so the fluorescence intensity was unchanged. It was proved that NRs-induced cell autophagy is a complete process.

10.5 Testing the ubiquitination dependence of NRs for degradation of mutant p53

ES-2 cells were seeded in a 24-well cell culture plate (density of about 5×10⁴ cells/well) and cultured overnight. PBS, NRs with a concentration of 500 μg/mL, PYR-41 (5 μM) and 500 μg/mL NRs+PYR-41 (5 UM) were added respectively. After 12 hours of culture, western blot detection was performed, and the results are shown in FIG. 19. FIG. 19 is a western blot image for detecting the ubiquitination dependence of NRs in degrading mutant p53 protein in ES-2 cells.

It can be seen from FIG. 19 that the ubiquitinase inhibitor PYR-41 can effectively inhibit the degradation of mutant p53 protein caused by NRs, indicating that the degradation of mutant p53 protein by NRs depends on the ubiquitination modification of mutant p53 protein.

10.6 Testing the effect of degradation mutant p53 by NRs on protein ubiquitination

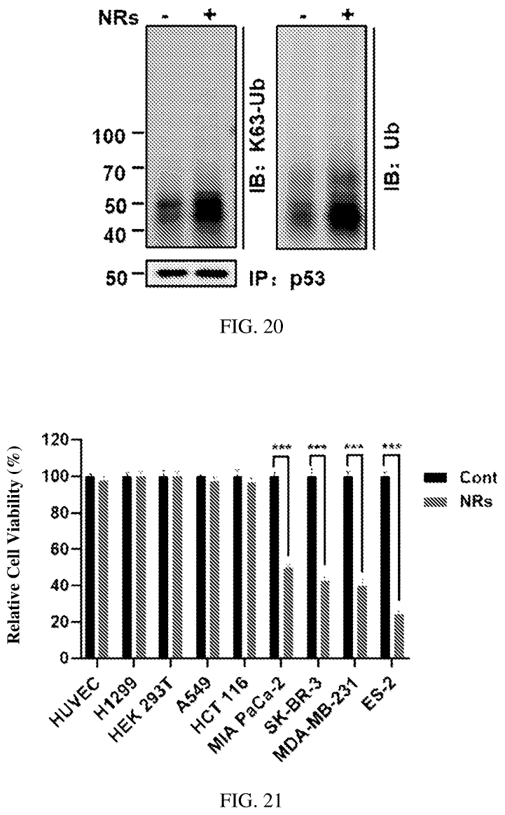

HEK 293T cells were seeded in a 6-well cell culture plate (density approximately 5×10⁵ cells/well) and cultured overnight. PBS or NRs with a concentration of 500 μg/mL was added. After culturing for 12 hours, the cells were lysed using IP lysis buffer, and cell extracts containing equal amounts of protein were incubated overnight with protein A/G-Sepharose and p53 antibodies (2 μg each). After washing with PBST buffer, the precipitate was separated on SDS-PAGE, and western blot was analyzed by using Ub antibody and K63-Ub antibody. The results are shown in FIG. 20. FIG. 20 is a western blot image of increased ubiquitination levels induced by NRs in ES-2 cells.

It can be seen from FIG. 20 that compared with the control group, the overall ubiquitination and K63 ubiquitination levels of mutant p53 protein in cells after NRs treatment were significantly increased.

11. NRs degrading mutant p53 inhibits the Gain-of-function (GOF) of mutant p53

### 11.1 Testing the Effect of NRs on the Viability of Various Cells

Normal cells (HUVEC and HEK 293T), tumor cells not expressing p53 (H1299), tumor cells expressing wild-type p53 protein (A549 and HCT 116), and tumor cells expressing mutant p53 protein (MIAPaCa-2, SK-BR-3. MDA-MB-231 and ES-2) were seeded in a 96-well cell culture plate (density approximately 1×10⁴/well) respectively, and cultured overnight. PBS and 500 μg/mL NRs were added. After culturing for 24 hours, MTT assay was performed, and the results are shown in FIG. 21. FIG. 21 shows the cell viability of HUVEC, H1299, HEK 293T, A549, HCT 116, MIAPaCa-2, SK-BR-3, MDA-MB-231 and ES-2 after NRs treatment.

It can be seen from FIG. 21 that NRs exhibited minimal effects on the viability of normal cells and tumor cells that do not express p53 and tumor cells that express wild-type p53 protein, but specifically reduced the viability of tumor cells that express mutant p53 protein, among which the effect of reducing cell viability in ES-2 cells was most obvious.

11.2 Testing the Evaluation of Free MBP, mNRs, dNRs and NRs on the Proliferation Ability of ES-2 Cells

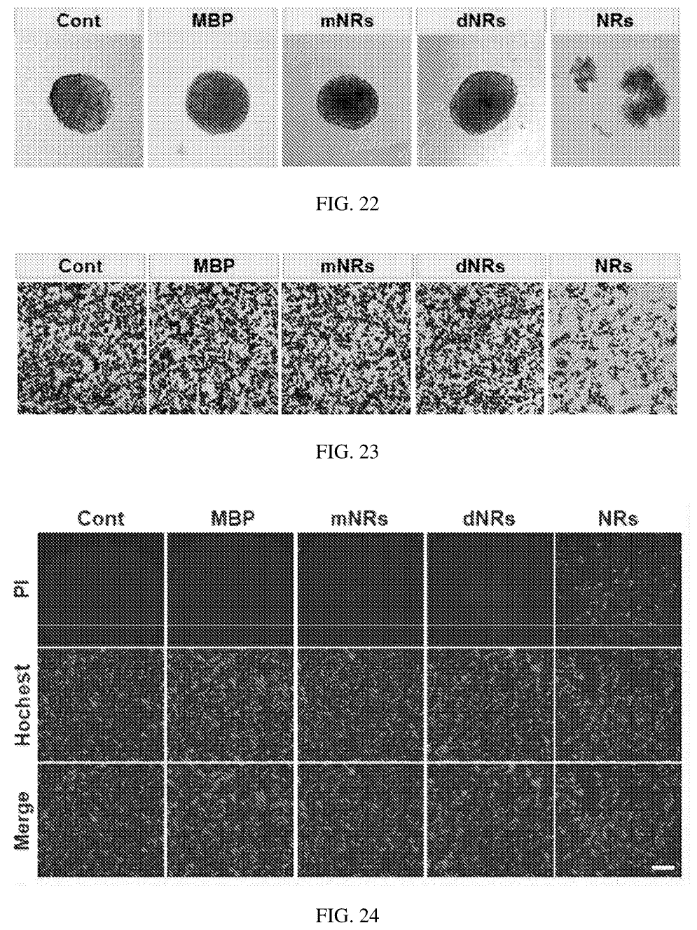

ES-2 cells were seeded into a 24-well low-adhesion cell culture plate containing serum-free medium (density 1×10³/well). 2% B27, basic fibroblast growth factor (20 ng/ml) and epidermal growth factor (20 ng/mL) were added. PBS, free MBP peptide (250 μg/mL), mNRs (500 μg/mL), dNRs (500 μg/mL) and NRs (500 μg/mL) were added, respectively. After 12 hours of culture, they were removed and replaced with fresh medium. After 7 days of culture, an inverted phase contrast microscope was used to record the diameters of the spheroids in different groups, and the results are shown in FIG. 22. FIG. 22 is a graph showing the sphering experiments of MBP, mNRs, dNRs and NRs in ES-2 cells.

It can be seen from FIG. 22 that compared with other treatment groups, the diameter of the cell spheroids after NRs treatment was significantly smaller, indicating that NRs can effectively inhibit the proliferation ability of ES-2 cells.

11.3 Testing the evaluation of free MBP peptides, mNRs, dNRs and NRs on the migration ability of ES-2 cells

ES-2 cells were seeded into the upper chamber in serum-free medium (density approximately 5×10³/well), and medium containing 10% FBS was added to the lower chamber. PBS, free MBP peptide (250 μg/mL), mNRs (500 μg/mL), dNRs (500 μg/mL) and NRs (500 μg/mL) were added to the upper chamber. After incubation for 12 hours, cells that migrated to the lower surface of the membrane were fixed with methanol and stained with 0.1% crystal violet. After washing with PBS three times, the cells were imaged microscopically, and the results are shown in FIG. 23. FIG. 23 is a graph showing Transwell results of MBP, mNRs, dNRs and NRs in ES-2 cells.

It can be seen from FIG. 23 that compared with other treatment groups, after NRs treatment, the number of cells migrating to the lower surface of the membrane was significantly reduced, indicating that NRs can effectively inhibit the migration ability of ES-2 cells.

11.4 Testing the evaluation of free MBP, mNRs, dNRs and NRs on the death ability of ES-2 cells

ES-2 cells were seeded in a 24-well cell culture plate (density approximately 5×10³/well), and cultured overnight. PBS, free MBP peptide (250 μg/mL), and mNRs, dNRs, and NRs (all have a concentration of 500 μg/mL) were added, respectively. After 24 hours of culture, Hoechst (10 μg/mL) dye was used to stain the cell nuclei, and propidium iodide (10 μg/mL) was used to stain the dead cells, and then the cells were detected by using a fluorescence microscope. The results are shown in FIG. 24. FIG. 24 is the Hoechst/PI double staining graph of MBP peptide, mNRs, dNRs and NRs in ES-2 cells.

It can be seen from that FIG. 24 compared with other treatment groups, the red fluorescence in cells treated with NRs was enhanced, indicating that NRs can effectively trigger ES-2 cell death.

12. The therapeutic effect at the animal level of degrading mutant p53 by NRs 12.1 In vivo biodistribution experiments

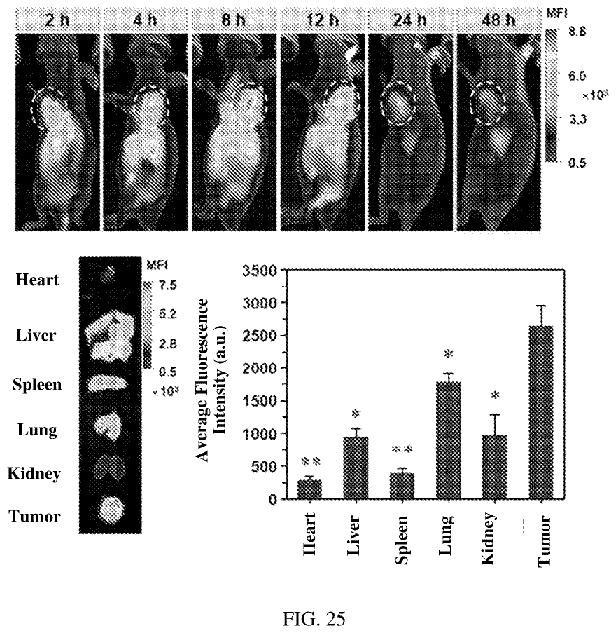

1×10⁷ ES-2 cells were subcutaneously injected into the right flank of BALB/c nude mice to construct an ES-2 tumor-bearing nude mouse model. When the tumor volume of the mice reached about 100 mm³, BALB/c nude mice carrying ES-2 xenografts were intravenously (i.v.) injected with 3 mg of DiD fluorescent dye-labeled NRs/Pt (DID-NRs/Pt, refer to the preparation method of NRs/Pt(IV) in Example 2, DiD fluorescent dye was added during the preparation of nanoparticles to obtain DiD-labeled DiD-NRs/Pt (IV)), and then imaged by Xenogen IVIS Lumina system at 2, 4, 8, 12, 24 and 48 hours. The mice were sacrificed 48 hours after the injection, and the main organs and tumor tissues were collected for photography and fluorescence statistical analysis. The results are shown in FIG. 25. Among them, FIG. 25 is the biodistribution graph of DID-NRs in ES-2 tumor-bearing mice.

It can be seen from FIG. 25 that the fluorescence intensity of nanoparticles in the mouse tumor site was gradually increased and then decreased over time, with the highest fluorescence intensity at 8 hours. Fluorescence images and statistical analysis of major organs and tumors showed that the average fluorescence intensity of NRs/Pt in the tumor site was the strongest, indicating that NRs/Pt can be effectively enriched in the tumor site.

### 12.2 In Vivo Anti-Tumor Treatment Experiments

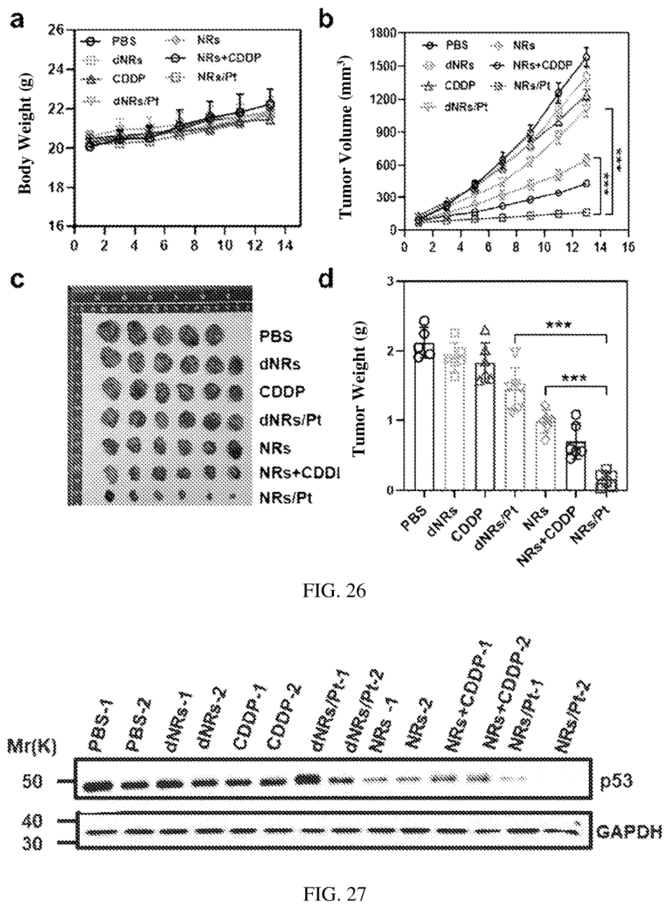

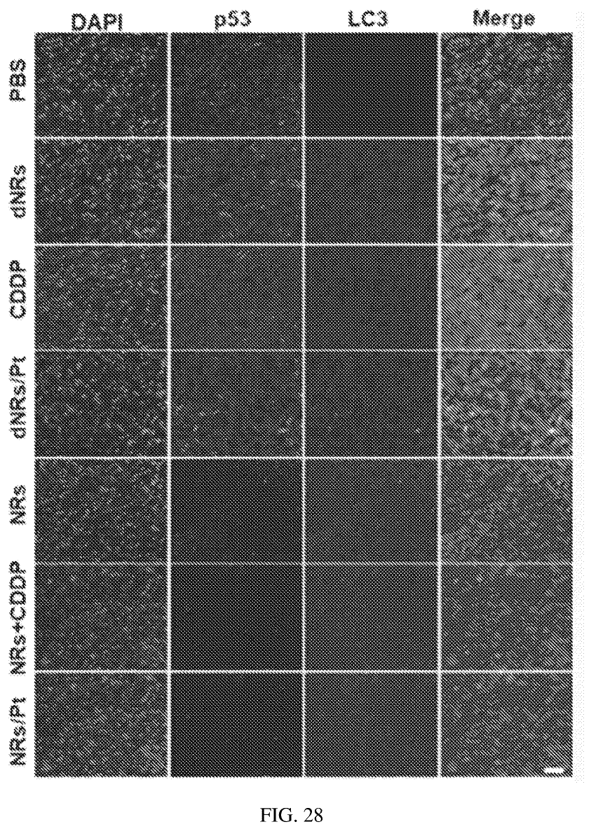

In order to explore the tumor-suppressing effect of nanoparticles in vivo, we established a human ES-2 ovarian cancer model in BALB/c nude mice subcutaneously. When the tumor volume reached about 100 mm³, the mice were randomly divided into the following 7 groups, with 6 mice in each group, namely: the PBS group, the dNRs group, the CDDP (cisplatin) group, the dNRs/Pt(IV) group, the NRs group, the NRs+CDDP group and the NRs/Pt(IV) group, where the equivalent injection doses of platinum, dNRs and NRs were 1.0 mg/kg, 25 mg/kg and 25 mg/kg, respectively. The drug was administered every other day, and the tumor volume and body weight of the mice were recorded. On the 13th day of treatment, the mice were sacrificed and the tumor tissues were removed and weighed, and the levels of mutant p53 protein and autophagy-related protein LC3 in the tumor tissues were detected. The results are shown in FIGS. 26, 27 and 28. FIG. 26 is a graph of the in vivo treatment experiment of nanoparticles in ES-2 tumor-bearing mice; FIG. 27 is a western blot image of p53 in tumors of each group of mice in ES-2 tumor-bearing mice; FIG. 28 is an immunofluorescence graph of p53 and LC3 in tumor sections of each group of mice in ES-2 tumor-bearing mice.

It can be seen from FIG. 26a that during the entire treatment process, the weight of mice in each group did not change significantly, proving that the components of each experimental group does not cause significant systemic toxicity to mice, and also reflecting that NRs has good biocompatibility. The mouse tumor growth curve in FIG. 26b and the in vitro tumor image in FIG. 26c show that compared with the PBS group, the dNRs group, the CDDP group and the dNRs/Pt(IV) group, the NRs group and the NRs+CDDP group can effectively inhibit tumor growth, and the NRs/Pt(IV) group can further inhibit the growth of tumor volume. In addition, being consistent with the tumor volume data, the tumor weights of mice in each group in FIG. 26d show that compared with other treatment groups, the NRs and chemotherapy drug CDDP combined treatment group effectively inhibited tumor growth, and NRs/Pt(IV) further reduced the weight of the tumor. FIGS. 27 and 28 show that, being consistent with the results obtained in the cell lines, the level of cellular autophagy in the tumor tissues of mice treated with NRs (including NRs, NRs+CDDP and NRs/Pt groups) was significantly enhanced, and the level of mutant p53 protein was significantly reduced.

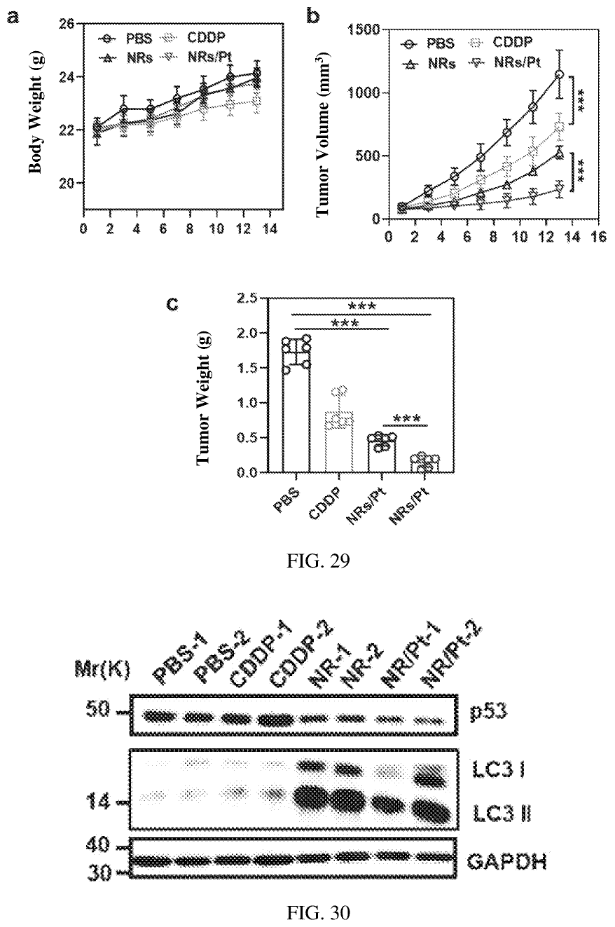

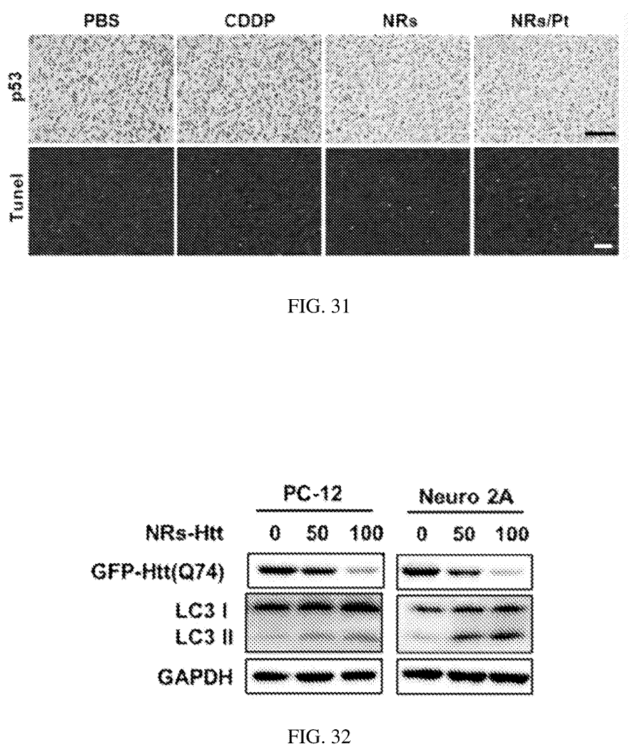

At the same time, in order to preliminarily understand the potential clinical effects of NRs, patient-derived p53 mutant (P72R+/+, C141Y+/+ and L350P+/−) ovarian cancer samples were transplanted subcutaneously into NOD/SCID mice to construct a PDX ovarian cancer model. When the tumor volume reached about 100 mm³, the mice were randomly divided into 4 groups, each with 6 mice. PBS, CDDP, NRs or NRs/Pt were injected into the mice through the tail vein, wherein the doses of platinum and NRs were 10 mg/kg and 25 mg/kg, respectively. The drug was administered once every two days, and the weight and tumor volume of the mice were recorded. On the 13th day of treatment, the mice were sacrificed, the tumor tissues were removed and weighed. The levels of mutant p53 protein and autophagy-related protein LC3 in the tumor tissues were detected. The results are shown in FIGS. 29, 30 and 31. FIG. 29 is a graph of the in vivo treatment experiment of nanoparticles in PDX model mice; FIG. 30 is a western blot image of p53 in the tumors of each group of mice in PDX model mice; and FIG. 31 is an immunohistochemistry and TUNEL staining graph of p53 in the tumor sections of each group of mice in PDX model mice.

As shown in FIG. 29a, the weight of mice in the CDDP group was slightly lower than that of other mice, but there was no significant difference in the weight of mice among the four treatment groups, which preliminarily indicated that the various treatments had no obvious toxic side effects on mice. In the mouse tumor growth curve in FIG. 29b, we found that the tumor volume of mice in the PBS-treated group was as high as 1000 mm² at the end of treatment, while the tumor volume growth of mice in the NRs-treated group and the CDDP-treated group was significantly slowed down, and the average tumor volume of mice in the NRs/Pt-treated group was only about 300 mm², proving that NRs/Pt further enhanced the tumor inhibition effect of NRs nanoparticles. Consistently, the tumor weight graph of each group of mice in FIG. 29c showed that compared with the PBS group, the tumor weight of the NRs-treated group alone was significantly reduced, and the tumor weight of the NRs/Pt group was further reduced.

As shown in FIG. 30, the NRs-treated group and the NRs/Pt-treated group effectively reduced the level of mutant p53 protein in the tumor tissue of PDX model mice, and at the same time enhanced the level of autophagy in the tumor tissue; finally, the results of immunohistochemistry and TUNEL staining of tumor tissue sections in FIG. 31 showed that the levels of mutant p53 protein in the tumor tissue of the NRs-treated group and the NRs/Pt(IV)-treated group were significantly reduced, while the levels of cell apoptosis were significantly enhanced.

### 13. Experiment of the Degradation of Mutant Huntingtin Protein Induced by NRs-Htt

PC-12 and Neuro 2A cells were seeded in a 24-well cell culture plate (density approximately 5×10⁴/well), and cultured overnight. Lipofectamine 3000 transfection reagent was used to transfect GFP-Htt (Q74) plasmid into PC-12 and Neuro 2A cells respectively, to obtain PC-12/GFP-Htt (Q74) and Neuro 2A/GFP-Htt (Q74) cells stably expressing GFP-Htt (Q74) mutant huntingtin protein.

The above two types of cells were seeded in 24-well cell culture plates (density of about 3×10⁴/well), and cultured overnight. PBS and NRs-Htt (50 or 100 g/mL) were added respectively. After culturing for 36 hours, western blot was performed to detect the levels of mutant huntingtin protein and autophagy-related LC3 protein. The results are shown in FIG. 32. FIG. 32 is a western blot image of NRs-Htt inducing cell autophagy effect and degrading GFP-Htt (Q74) protein in PC-12/GFP-Htt (Q74) and Neuro 2A/GFP-Htt (Q74) cells;

It can be seen from FIG. 32 that NRs-Htt can effectively enhance the level of autophagy-related LC3II protein, induce cell autophagy, and reduce the level of mutant huntingtin protein in PC-12/GFP-Htt (Q74) and Neuro 2A/GFP-Htt (Q74) cells.

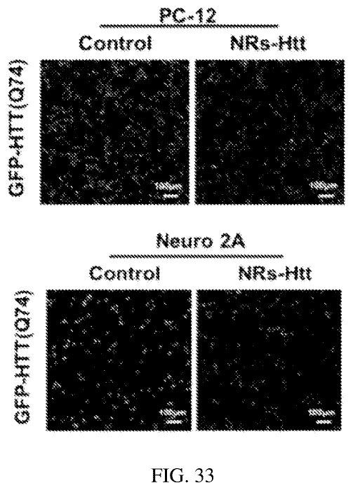

The above two cells were seeded in 24-well cell culture plates (density of about 3×10⁴/well) with circular slides in advance, and cultured overnight. PBS and NRs-Htt (100 μg/mL) were added respectively. After culturing for 36 hours, immunofluorescence detection was performed. The results are shown in FIG. 33. FIG. 33 is an immunofluorescence image of degrading GFP-Htt (Q74) protein by NRs-Htt in PC-12/GFP-Htt (Q74) and Neuro 2A/GFP-Htt (Q74) cells.

As shown in FIG. 33, NRs-Htt can effectively reduce the level of mutant huntingtin protein in PC-12/GFP-Htt (Q74) and Neuro 2A/GFP-Htt (Q74) cells.

The above embodiments are only used to illustrate the technical solution of the present application, but not to limit the present application; although the present application is described in detail with reference to the above embodiments, a person skilled in the art should understand that the technical solution described in the above embodiments can still be modified, or part or all of the technical features thereof can be replaced by equivalents; and these modifications or replacements do not make the essence of the corresponding technical solution deviate from the scope of the technical solution of the present application.

What is claimed is:

1. A nanocomposite for targeted degradation of a pathogenic protein, wherein the nanocomposite contains a nanocarrier and a pathogenic protein-targeting binding peptide grafted on the nanocarrier;
wherein the nanocarrier is a nanoassembly of maleimide-polyethylene glycol-polylactic acid and cationic lipids.

2. The nanocomposite of claim 1, wherein the cationic lipids includes 1,2-dioleoyl-3-trimethylammonium-propane, 2,3-dioleoyloxypropyl-1-trimethylammonium bromide.

3. The nanocomposite of claim 1, wherein the mass ratio of the cationic lipids to the maleimide-polyethylene glycol-polylactic acid is (5-40): 100.

4. The nanocomposite according to of claim 1, wherein the pathogenic protein includes mutant p53 protein and mutant huntingtin protein;
the mutant p53 protein contains at least one mutation site selected from the group consisting of S241F, R175H, R248W, and R280K mutation sites; and
the mutant huntingtin protein includes Htt-(Q74) mutation type.

5. The nanocomposite of claim 4, wherein the mutant p53 protein-targeting binding peptide has an amino acid sequence of SEQ ID NO.1; and
the mutant huntingtin protein-targeting binding peptide has an amino acid sequence of SEQ ID NO.2.

6. A method for preparing a nanocomposite for targeted degradation of a pathogenic protein, the nanocomposite containing a nanocarrier and a pathogenic protein-targeting binding peptide grafted on the nanocarrier, wherein the method includes:
subjecting the pathogenic protein-targeting binding peptide to undergo a click reaction with a solution containing the nanocarrier, and performing dialysis, ultrafiltration and concentration on the reaction product in sequence to obtain the nanocomposite for targeted degradation of the pathogenic protein.

7. The method of claim 6, wherein the molar ratio of the pathogenic protein-targeting binding peptide to the nanocarrier is (1-3): 1.

8-10: (canceled)

11. The method claim 6, wherein the cationic lipids includes 1,2-dioleoyl-3-trimethylammonium-propane, 2,3-dioleoyloxypropyl-1-trimethylammonium bromide.

12. The method of claim 6, wherein the mass ratio of the cationic lipids to the maleimide-polyethylene glycol-polylactic acid is (5-40): 100.

13. The method of claim 6, wherein the pathogenic protein includes mutant p53 protein and mutant huntingtin protein;
the mutant p53 protein contains at least one mutation site selected from the group consisting of S241F, R175H, R248W, and R280K mutation sites; the mutant huntingtin protein includes Htt-(Q74) mutation type.

14. The method of claim 13, wherein the mutant p53 protein-targeting binding peptide has an amino acid sequence of SEQ ID NO.1; the mutant huntingtin protein-targeting binding peptide has an amino acid sequence of SEQ ID NO.2.

15. The method of claim 6, further comprising:
preparing an anti-tumor nanodrug using the nanocomposite.

16. The method of claim 6, further comprising:
preparing a Huntington's disease inhibiting drug using the nanocomposite.

17. An anti-tumor nanodrug comprising:
a nanocomposite containing a nanocarrier and a pathogenic protein-targeting binding peptide grafted on the nanocarrier;
wherein the nanocarrier is a nanoassembly of maleimide-polyethylene glycol-polylactic acid and cationic lipids; and
anti-tumor active ingredient(s) loaded in the nanocomposite;
wherein, the anti-tumor active ingredient(s) include(s) cisplatin Pt(IV) prodrug.

18. The anti-tumor nanodrug of claim 17, wherein the cationic lipids includes 1,2-dioleoyl-3-trimethylammonium-propane, 2,3-dioleoyloxypropyl-1-trimethylammonium bromide.

19. The anti-tumor nanodrug of claim 17, wherein the mass ratio of the cationic lipids to the maleimide-polyethylene glycol-polylactic acid is (5-40): 100.

20. The anti-tumor nanodrug of claim 17, wherein the pathogenic protein includes mutant p53 protein and mutant huntingtin protein;
the mutant p53 protein contains at least one mutation site selected from the group consisting of S241F, R175H, R248W, and R280K mutation sites; and
the mutant huntingtin protein includes Htt-(Q74) mutation type.

21. The anti-tumor nanodrug of claim 20, wherein the mutant p53 protein-targeting binding peptide has an amino acid sequence of SEQ ID NO.1; and
the mutant huntingtin protein-targeting binding peptide has an amino acid sequence of SEQ ID NO.2.

22. The anti-tumor nanodrug of claim 17, wherein the anti-tumor nanodrug is formed by:
self-assembling a solution containing maleimide-polyethylene glycol-polylactic acid, cationic lipids and anti-tumor active ingredient(s) in a buffer to obtain a solution containing drug-loaded nanoparticles; and
subjecting the solution containing drug-loaded nanoparticles to undergo a click reaction with the protein-targeting binding peptide, and performing dialysis, ultrafiltration and concentration on the reaction product in sequence to obtain the anti-tumor nanodrug.
